(19) United States
(12) Patent Application Publication
(10) Pub. No.: US 2014/0024889 A1
(43) Pub. Date: Jan. 23, 2014

(54) Gaze Contingent Control System for a Robotic Laparoscope Holder

(71) Applicant: Wilkes University, Wilkes-Barre, PA (US)

(72) Inventor: Zhang Xiaoli, Wilkes-Barre, PA (US)

(21) Appl. No.: 13/941,632

(22) Filed: Jul. 15, 2013

Related U.S. Application Data
(60) Provisional application No. 61/672,322, filed on Jul. 17, 2012.

(51) Int. Cl. A61B 1/00; A61B 19/00; A61B 1/313

(52) U.S. Cl. CPC A61B 1/00039; A61B 1/00149; A61B 1/3132; A61B 19/2203; A61B 1/00045
USPC 600/102

(57) ABSTRACT

A gaze contingent control system for a robotic laparoscope holder which has a video-based remote eye tracking device and at least one processor capable of receiving eye gaze data from said eye tracking device and in response outputting a series of control signals for moving said robotic laparoscope.

### CROSS-REFERENCE TO RELATED APPLICATIONS

This application claims priority under 35 U.S.C. §119(e)(1) from U.S. Provisional Patent Application No. 61/672,322, filed on Jul. 17, 2012, for “Gaze Contingent Control System for a Robotic Laparoscope Holder,” the disclosure of which is incorporated herein by reference.

### STATEMENT REGARDING FEDERALLY SPONSORED RESEARCH

Not Applicable.

### BACKGROUND

1. Field of Invention

The present invention relates to eye-movement-based robot control. In particular, the present invention relates to an eye-tracking system that allows a surgeon to control an assistive robotic laparoscope holder.

2. Description of Related Art

Laparoscopic surgery is well known in modern medical practice. Typically, laparoscopic surgery involves the use of surgical tools (e.g., clamps, scissors) that are attached to the end of extended instruments which are designed to be inserted through a small incision and then operated inside a patient's body together with a laparoscope that allows the surgeon to see the surgical field on a monitor. Common laparoscopic surgeries include cholecystectomy, colectomy, and nephrectomy.

One problem inherent with known laparoscopic surgery techniques is the surgeon's lack of control over the laparoscope. Typically, the laparoscope is controlled by an assistant. As the surgeon uses both of his/her hands to manipulate the instruments, he/she must verbally communicate with the assistant whenever a new segment of the surgical field needs to be seen. In light of the fact that the assistant is positioned in a different point of reference in relation to the surgeon and the surgical field is being projected remotely from the patient's body, it can be difficult for the assistant to fully understand which are of the surgical field the surgeon would like to view/focus on.

To solve this problem, robot-assisted laparoscope holders were introduced. An example of such a holder is the automated laparoscope system for optimal positioning (AESOP) which can be controlled with pre-calibrated voice commands. Another example is the EndoAssist from Armstrong Healthcare Ltd. The EndoAssist is controlled by the surgeon's head movement via infrared emitters that communicate with a sensor placed above a monitor. A foot clutch is used to engage and disengage the robotic holder so the surgeon can control when it moves to a different location and when it does not.

While these examples remove the need for a human assistant, voice-recognition and head controls still require the surgeon's physical interventions in laparoscope manipulation, which create other problems. These interventions in laparoscope adjustments are obtrusive barriers for the surgeon to naturally and intuitively visualize the surgical site. Voice-recognition software may accept or interpret the wrong command and may limit what a surgeon can say to others in the operating room so as not to misdirect the robotic holder. Having to move his/her head while performing surgery may cause the surgeon to look away from the surgical field momentarily in order to direct the robotic holder—an action that may complicate the surgery or pose risk to the patient due to the fact that may laparoscopic surgeries take place in confined cavities within the body and involve or occur adjacent to vital organs. Similarly, frequent head movements may tire the surgeon, especially during multiple-hour surgeries.

Accordingly, there is a need for a system that will enable a surgeon to perform a laparoscopic surgery without a human assistant and in such a way that minimizes the risk of error and physical exertion of the surgeon.

### SUMMARY

The present invention is a system that allows a surgeon to control a robot-assisted laparoscope holder with his/her eye gaze. The system comprises a robot-assisted laparoscope holder that is networked with an eye tracking system by a microprocessor running the commercial software program LABVIEW™. The eye tracking system is a video-based tracking system with cameras and infrared lights.

### BRIEF DESCRIPTION OF THE DRAWINGS

FIG. 1 is a side view of a system consistent with the present invention.

FIG. 2 is a flow chart describing one embodiment of the present invention.

### DETAILED DESCRIPTION

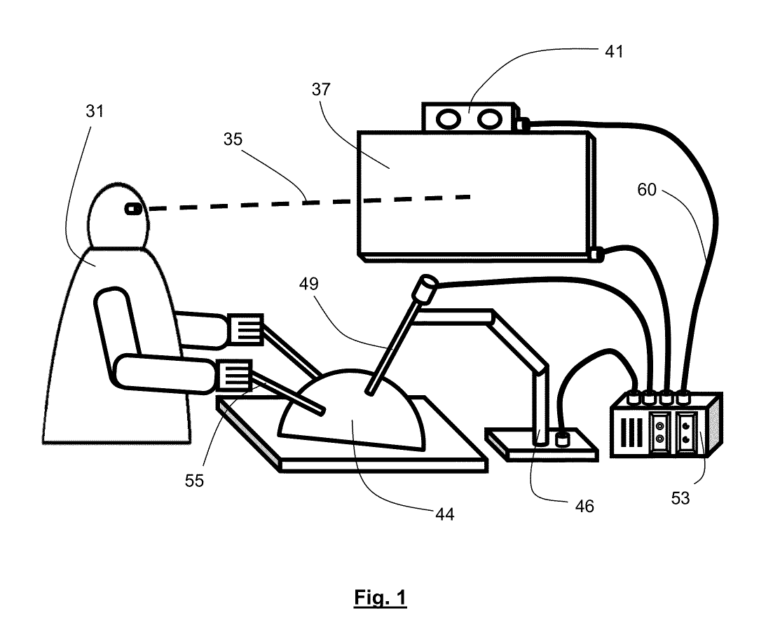

The purpose of the invention in all of its embodiments is to provide a system that allows a surgeon to control a robot-assisted laparoscope holder with his/her eye gaze. As is shown in FIG. 1, the system comprises a robot-assisted laparoscope holder 46 that is networked 60 with an eye tracking system. The eye tracking system comprises a display 37 and an eye-gaze-tracking sensor 41. When in use, the surgeon 31 gazes 35 at the display 37, which is broadcasting a video feed from the laparoscope 49 though the system's network 60. As the surgeon 31 fixes his/her gaze 35 on different areas of the display 37, the eye-gaze-tracking sensor 41 tracks the gaze 35 and sends coordinate information through the system's network 60 to a microprocessor 53. The microprocessor 53 then processes the information it receives from the eye-gaze-tracking sensor 41, via a commercially-available software program called LABVIEW™, and determines instructions to submit to the robot-assisted laparoscope holder 46. If the laparoscope 49 is to be moved in order to correspond with movement of the surgeon's 31 eye gaze 35, the microprocessor will instruct the robot-assisted laparoscope holder 46 to move the laparoscope 49 accordingly. As the robot-assisted laparoscope holder 46 moves the laparoscope 49, the view being broadcast on the display 37 changes until the desired location of the surgical field comes into view. As a result, the surgeon 31 is able to change the view of the surgical field without having to remove his/her hands from the surgical tools 55 being used in the patient 44.

In preferred embodiments of the invention, the robot-assisted laparoscope holder is a CoBRASurge robot as disclosed in U.S. Pat. No. 8,282,653 (incorporated herein by reference). CoBRASurge creates a mechanically constrained remote center of motion (“RCM”) with three rotational degrees of freedom (“DOFs”) about the rotation center and one translational DOF passing through it. The rotation center would coincide with the surgical entry port during the surgery. The laparoscope can be fitted into the articulated mechanism using a collar. It can produce a cone workspace with 60 vertex angle and its tip locates at the incision port. There are four motors mounted on CoBRASurge, three for orientation about the center of RCM and one for the insertion-extraction of the laparoscope. In preferred embodiments of the present invention, a webcam with high resolution 1600×896 is mounted on a slender shaft acting as a laparoscope.

In a preferred embodiment of the present invention, a S2 Eye Tracker from Mirametrix (“S2”) is used as the eye-gaze-tracking sensor. S2 is a video-based remote eye tracking system that allows a certain amount of head movement within a working volume of 25×11×30 cm³. S2 can report the gaze data at 60 Hz with an accuracy of 0.5°-1° and draft <0.3°; An advanced calibration is needed before it can be used for tracking Raw gaze data is analyzed to obtain a stable gaze position before transmission to the microprocessor and corresponding laparoscope control software.

The performance of the eye tracker system depends greatly on the initial calibration, which builds the correlation between eye movements and gaze positions on the display. The surgeon sits in a comfortable position in front of the display where the eye-gaze-tracking sensor can successfully track the surgeon's eye gaze when he/she looks at discretional positions on the display. In the calibration process, nine (9) shrinking circles are displayed on the display consecutively, which keep shrinking to a point and then disappear. And the surgeon is asked to stare at each circle when it is showing. At the end of the calibration, the system estimates the calibration performance, and a curser displays on the display indicating the current gaze position of the eyes.

Based on an advanced calibration of the S2, it can give out the position where the surgeon is looking at, referring to gaze position. The raw gaze position data is refined first before being used to determine a fixation. The refinement and fixation determine processes are as follows:
1) Check if the new reported gaze point falls outside the tracking window (0-1); yes, discard it and wait for the next one; no, go to step 2.
2) Check if the new reported gaze point is within a circle with std (standard deviation) as radius centers at the average of queue A storing last several points, then update queue
   A:
      a) Within the range, restore the new data and keep the size of queue A not larger than 10 and go to step 3.
      b) Outside the range, if A is not empty discard it and the first point in the queue; else restore the new point. Then go to step 3.
3) Calculate the average of queue A and restore it with previous averages in queue B, go to step 4.
4) Check the size of queue B if it is larger than 80; yes, go to step 6; no go to step 1.
5) Check if 75% of the points in queue B are within a circle taking 80 pixels as radius and the mean as center; yes, fixations is obtained, then refresh the queue B and go to step 1; no, refresh the queue B and go to step 1.

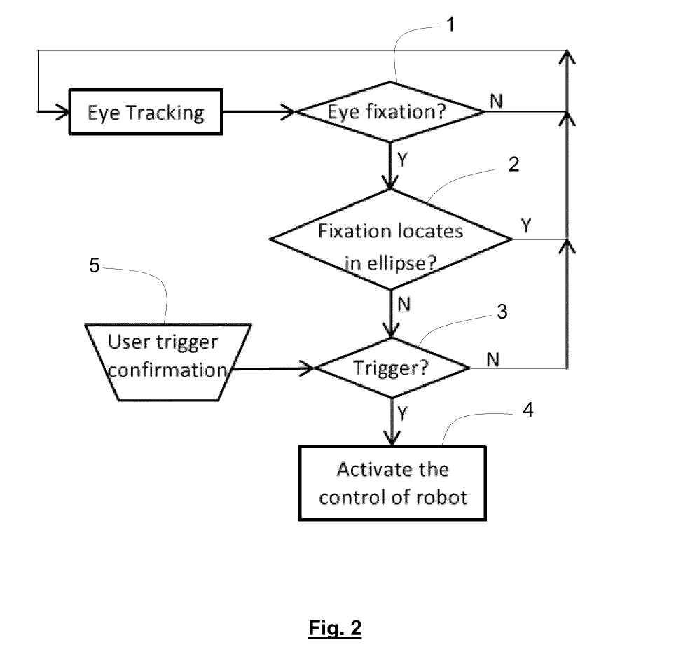

When a direction vector is reported in the image coordinate system, it can be translated to the robot-base coordinate system. This process is illustrated in FIG. 2. After the robot has been activated, the stabilized gaze and the surgeon's fixation 1 are transferred to the controller of the robot. The deviation from the center of the display 2 to the fixation indicates 3 the direction and the travel distance that CoBRASurge needs to move along after a transformation 4. Meanwhile the drift between stabilized gaze point and the display center is taken as the reference whether the stared object has moved to the center of the field-of-view. On the display, an elliptical area at the center is defined as the surviving area. When the surgeon's gaze position falls into the elliptical area, the CoBRASurge stands still to keep the current focus view until next trigger signal. When the reported fixation locates outside of the area, it is shown on the screen to the surgeon for checking As an additional safety precaution, a physical confirmation 5 is provided to the user to determine whether it is the user's intention to take the current fixation to activate the robot. This physical confirmation could be, for example, a foot clutch (or similar device) with three pedals, one for trigger confirmation and the other two for zooming in and out, or the space bar, left and right buttons on a keyboard. Once the user confirms the intended trigger the robot is activated and attempts to head the laparoscope to the interested object and guide it locate at the center of the field-of-view. The position where the laparoscope axial shaft is perpendicular to the horizontal plane (patient body or patient table) is taken as the default position.

The drift between the gaze position and screen center is processed with a reduction factor before it is transferred to the robot's motion commands. Since the laparoscope loses the perception of the depth, the pixel distance on the display image may indicate various travel distance commands to the robot. To solve this problem, we introduce a reduction factor, which is proportional to the intervention depth of the laparoscope. When camera is at the top of the abdomen, the intervention is recorded and the corresponding travel distance for the robot is properly determined. If the maximal intervention depth is D, and the current intervention depth is d, the reduction factor can be calculated by (D-d)/D. The extreme condition, in which the camera is extremely close to the targets, is ignored.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A gaze contingent control system for a robotic laparoscope holder comprising:
(a) a robotic laparoscope;
(b) a video-based remote eye tracking device; and
(c) at least one processor capable of receiving eye gaze data from said eye tracking device and in response outputting a series of control signals for moving said robotic laparoscope.
